(19) United States
(12) Patent Application Publication
(10) Pub. No.: US 2022/0031580 A1
(43) Pub. Date: Feb. 3, 2022

(54) GEL COMPOSITIONS COMPRISING HYDROPHOBIC SILICA AND A COPOLYMER FORMED FROM SULFONIC ACID ACRYLIC MONOMER

(71) Applicant: L'OREAL, Paris (FR)

(72) Inventors: Gloria LOPEZ, Fairview, NJ (US); Sonal Patel, Iselin, NJ (US); Ramakrishnan Hariharan, Springfield, NJ (US); I-Fan Hsieh, Scotch Plains, NJ (US)

(73) Assignee: L'OREAL, Paris (FR)

(21) Appl. No.: 16/944,898

(22) Filed: Jul. 31, 2020

(51) Int. Cl. A61K 8/04; A61K 8/25; A61K 8/81; A61K 8/87

(52) U.S. Cl. CPC A61K 8/042; A61K 8/25; A61K 8/87; A61K 8/8188; A61K 8/8152

(57) ABSTRACT

Gel compositions including water, a water-soluble or water-dispersible gelling agent comprising at least one copolymer formed from sulfonic acid acrylic monomer are provided. The compositions further include a film-forming polymer selected from a film-forming acrylic polymer, a film-forming urethane polymer, and combinations thereof. The compositions further include hydrophobic silica and are substantially free of fatty compounds.

### FIELD OF THE INVENTION

The present invention relates to gel compositions comprising hydrophobic silica and copolymer formed from sulfonic acid acrylic monomer. Among other improved or beneficial properties, these compositions have surprisingly good properties including phase stability and water resistance.

### DISCUSSION OF THE BACKGROUND

Makeup compositions, particularly those useful for foundations and facial primers are difficult to formulate, especially for use in humid environments. It is challenging to create formulas that are comfortable, water repellant, phase stable, and that provide non-transfer properties. The present inventors have surprisingly found that by combining particular gelling agents and hydrophobic silica with film formers, an advantageous blend of properties is obtained.

### SUMMARY OF THE INVENTION

The present invention relates to gel compositions including water, a water-soluble or water-dispersible gelling agent comprising at least one copolymer formed from sulfonic acid acrylic monomer. The compositions further include a film-forming polymer selected from a film-forming acrylic polymer, a film-forming urethane polymer, and combinations thereof; and hydrophobic silica. The compositions are substantially free of fatty compounds. According to certain embodiments, the gel compositions include at least about 0.75% by weight of the at least one copolymer formed from sulfonic acid acrylic monomer.

The present invention also relates to methods of treating, caring for and/or making up keratinous materials by applying compositions of the present invention to a keratinous material in an amount sufficient to treat, care for and/or make up the keratinous material.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

All percentages of ingredients herein are listed on an actives basis unless specifically stated otherwise. Further, all percentages of ingredients are in percent by weight unless specifically stated otherwise.

Other than in the operating examples, or where otherwise indicated, all numbers expressing quantities of ingredients and/or reaction conditions are to be understood as being modified in all instances by the term “about,” meaning within 10% to 15% of the indicated number. For example, about 10% means from 8.5% to 11.5%, such as between 9% and 11%.

Numerical ranges are inclusive of endpoints and meant to include all combinations and sub-combinations. For example, from about 5%, 10% or 15% to about 20%, 50% or 60% means about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%.

All concentrations in this specification are by weight unless otherwise specifically stated differently.

“Film former” or “film forming agent” as used herein means any material such as, for example, a polymer or a resin that leaves a film on the substrate to which it is applied.

“Polymer” as used herein means a compound which is made up of at least two monomers.

“Keratinous materials” includes materials containing keratin such as hair, skin, eyebrows, lips and nails.

“Substantially free” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. In certain embodiments, substantially free means less than about 2% of the identified ingredient, such as less than about 1%, such as less than about 0.5%, such as less than about 0.1% of the ingredient. The term “anhydrous” means substantially free of water.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile”, as used herein, means having a flash point of less than about 100° C.

“Non-volatile”, as used herein, means having a flash point of greater than about 100° C.

“Long wear” compositions as used herein refers to compositions where the compositions, after application to a keratinous material, do not transfer or smudge after contact with another substrate and retain a consistent appearance on the keratinous material for an extended period of time. “Long wear” compositions, as used herein can also refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to keratinous materials such as skin, eyelashes or lips and evaluating the color of the composition after an extended period of time. For example, the color of a composition may be evaluated immediately following application to the keratinous material and these characteristics may then be re-evaluated and compared after a certain amount of time. Further, these characteristics may be evaluated with respect to other compositions, such as commercially available compositions. Additionally, long wear properties may be evaluated by applying a sample, allowing it to dry, and then abrading the sample to determine removal/loss of sample.

“Transfer” as used herein refers to the displacement of a fraction of a composition which has been applied to a keratinous material by contact with another substrate, whether of the same nature or of a different nature. For example, when a composition such as an eyeshadow, eyeliner or mascara has been applied, the composition can be transferred onto hands by rubbing or by contact of the hands with the eyes. By way of further example, when a composition such as a lipstick has been applied, the composition can be transferred onto teeth or hands, or onto the cheek of another person. Irrespective of composition type, the composition can also transfer from the keratinous material to which it has been applied to another substrate such as napkins, collars, glasses, cups or other containers.

“Transfer-resistance” as used herein refers to the quality exhibited by a composition in resisting transfer. To determine transfer-resistance, the amount of composition transferred from a keratinous material to a substrate may be evaluated and compared. For example, a composition may be transfer-resistant if, after application to a keratinous material such as lips, skin or eyelashes and contact with a substrate, a majority of the composition is left on the wearer. Further, the amount transferred may be compared with that transferred by other compositions, such as commercially-available compositions. In a preferred embodiment of the present invention, little or no composition is transferred to the substrate from the keratinous material.

“Oil-resistance” or “sebum-resistance” as used herein means transfer resistance when the composition contacts oil or sebum, respectively.

The compositions and methods of the present invention can comprise, consist of, or consist essentially of the essential elements and limitations of the invention described herein, as well as any additional or optional ingredients, components, or limitations described herein or otherwise useful in personal care.

In accordance with the present invention, gel compositions of the present invention include a gelling system that includes one or more copolymers of formed from sulfonic acid acrylic monomers.

Copolymer Formed from Sulfonic Acid Acrylic Monomer

In accordance with the present invention, compositions that include a gelling system that includes at least one copolymer formed from sulfonic acid acrylic monomer are provided.

Suitable copolymers formed from sulfonic acid acrylic monomers include crosslinked acrylamide/sodium acrylamido-2-methylpropanesulfonate (AMPS) copolymers, such as that used in the commercial product Sepigel 305 (CTFA name: Polyacrylamide/C₁₃-C₁₄ Isoparaffin/Laureth-7) or that used in the commercial product sold under the name Simulgel 600 (CTFA name: Acrylamide/Sodium Acryloyldimethyltaurate/Isohexadecane/Poly sorbate-80) by the company SEPPIC; copolymers of AMPS® and of vinylpyrrolidone or vinylformamide, such as that used in the commercial product sold under the name Aristoflex AVC® by the company Clariant (CTFA name: Ammonium acryloyldimethyltaurate/VP copolymer) but neutralized with sodium hydroxide or potassium hydroxide; copolymers of AMPS and of sodium acrylate, for instance the AMPS/sodium acrylate copolymer, such as that used in the commercial product sold under the name Simulgel EG® by the company SEPPIC or under the trade name Sepinov EM (CTFA name: Hydroxyethyl acrylate/sodium acryloyldimethyl taurate copolymer); copolymers of AMPS and of hydroxyethyl acrylate, for instance the AMPS/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyl taurate copolymer).

According to certain embodiments, compositions of the present invention include both (1) a copolymer of AMPS and of sodium acrylate and (2) a copolymer of AMPS and of hydroxyethyl acrylate.

The one or more copolymers formed from sulfonaic acid acrylic monomers may be present in an active content amount ranging from greater than 0.75% to about 20%, such as from about 0.75%, 1.0%, or 1.25% by weight to about 1.5%, 3%, or 5% by weight with respect to the total weight of the composition.

Film Forming Polymer

In accordance with the present invention, compositions include at least one film forming polymer. By “film-forming” polymer, it is meant a means polymer or resin that leaves a film (e.g., a continuous film) on the substrate to which it is applied, for example, after a solvent accompanying the film former has evaporated, absorbed into and/or dissipated on or from the substrate. According to certain embodiments, the film former when tested according to the following drawdown test forms a conformal coating and/or can picked up or scraped off with a razorblade to be removed as a free standing film. Five to ten grams of material is placed on the center of a Leneta card stock (Black and White Opacity card Chart 2812 available from BYK Additives and Instruments of Geretsried, Germany) and using a 3 mil Drawdown Birdbar (also from Byk), the material is spread across the sheet (8 in by 3 in) and allowed to dry overnight. Film-formers that are cosmetically or dermatologically acceptable may be utilized in the present invention. As used herein, “cosmetically acceptable” or “dermatologically acceptable” is intended to mean that a composition is suitable for use in contact with human tissues such as keratinous materials and mucous membranes without undue toxicity, incompatibility, instability, and/or allergic response.

The film-forming polymer is selected from a film-forming acrylic polymer, a film-forming urethane polymer, and combinations thereof. In certain notable embodiments, the gel compositions include both a film-forming acrylic polymer and a film-forming urethane polymer.

The film-forming polymer (total combined concentration of film forming acrylic polymer and film forming urethane polymer) is preferably present in the compositions of the present invention in an active solid content amount ranging from about 0.1%, 0.2%, 0.25% or 0.5% to about 2%, 3%, 4%, 10%, 20% or 25% by weight with respect to the total weight of the composition.

By “acrylic,” it is meant that the film forming polymer, is formed from the polymerization, for example, the free radical polymerization, of one or more ethylenically unsaturated monomers namely a vinyl, (meth)acrylic or allylic group. The term “(meth)acryl” and variations thereof, as used herein, means acryl or methacryl.

Suitable film forming acrylic polymers include those having acrylic acid, acrylamide, methacrylamide, alkyl- and dialkylacrylamide, alkyl- and dialkylmethacrylamide, alkyl acrylate, alkyl methacrylate monomers.

The film-forming acrylic polymer may be a latex. By “latex” is a colloidal dispersion of polymer particles in an aqueous liquid phase. “Latex” is generally obtained by suspension or emulsion polymerization or copolymerization of monomers according to processes that are well known to those of ordinary skill in the art. Such monomers may be chosen in particular from styrene, butadiene, acrylonitrile, chloroprene, vinyl acetate, urethanes, isoprene, isobutylene, and acrylic or methacrylic acid, maleic acid, crotonic acid or itaconic acid or esters or amides thereof. The latex may include a polymer chosen from copolymers resulting from the polymerization of at least one styrene monomer and at least one C₁-C₁₈ alkyl (meth)acrylate monomer, preferably C₁-C₁₂ alkyl (meth)acrylate, and preferably C₁-C₁₀ alkyl (meth)acrylate. The C₁-C₁₈ alkyl (meth)acrylate monomer is preferably chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate. As styrene monomer that may be used in the invention, examples that may be mentioned include styrene and α-methylstyrene.

Suitable examples of film forming acrylic polymers include EPITEX 66 polymer, an aqueous polyacrylate emulsion available from Dow Chemical and LUVIFLEX SOFT, available from BASF Corporation.

The film forming acrylic polymer is preferably present in the compositions of the present invention in an active solid content amount ranging from about 0.1% to about 20%, preferably from about 0.2% to about 10%, preferably from about 0.25% to about 5%, and preferably from about 0.33% to about 3%, by weight with respect to the total weight of the composition.

Film Forming Urethane Polymer

In accordance with the present invention, compositions include at least one film forming urethane polymer. By “urethane” polymer it is meant polymers formed from reacting isocyanates and polyols, and having a plurality of —NHCOO— linkages.

The film forming urethane polymer may exist as a dispersion or latex. Commercially available examples of such dispersions or latexes include, but are not limited to, aqueous polyurethane dispersions comprising a reaction product of a prepolymer comprising a dihydroxyl compound, a polyisocyanate, and a low molecular weight diol and at least two diamine compounds and wherein the composition is substantially free of triethanolamine stearate such as, for example, those sold under the BAYCUSAN® name by Bayer such as, for example, BAYCUSAN® C1000 (polyurethane-34), BAYCUSAN® C1001 (polyurethane-34), BAYCUSAN® C1003 (polyurethane-32), and BAYCUSAN® C1004 (polyurethane-35).

According to certain embodiments, the film forming urethane polymer is Polyurethane-34, such as polyurethane polymer dispersions sold under the Baycusan™ C1000 tradename from Bayer. It is sold as a 40% polymer solution.

The film forming urethane polymer is preferably present in the compositions of the present invention in an active solid content amount ranging from about 0.1% to about 20%, preferably from about 0.2% to about 10%, preferably from about 0.25% to about 5%, and preferably from about 0.33% to about 3%, by weight with respect to the total weight of the composition.

Hydrophobic Silica

The inventors have surprisingly found that hydrophobic silica, despite having no affinity for water, can be used in gel compositions of the present invention to enhance stability and/or water-resistance. Suitable hydrophobic silicas include, but are not limited to, silicas, such as pyrogenic silica with hydrophobic surface treatment whose particle size is less than 1 micron, preferably less than 500 nm, preferably less than 100 nm, preferably from 5 nm to 30 nm, including all ranges and subranges therebetween. It is in fact possible to modify the surface of silica chemically, by a chemical reaction producing a decrease in the number of silanol groups present on the surface of the silica. The silanol groups can notably be replaced with hydrophobic groups: a hydrophobic silica is then obtained. The hydrophobic groups can be:

trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812e” by the company Degussa, “CAB-O-SIL TS-530®” by the company Cabot; or “VM-2270 AEROGEL FINE PARTICLES” by Dow.

dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references; “AEROSIL R972®”, “AEROSIL R974®” by the company Degussa, “CAB-O-SIL TS-610e”, “CAB-O-SIL TS-720®” by the company Cabot.

Also, silica aerogel particles can be added to compositions of the present invention, if desired. Silica aerogels are porous materials obtained by replacing (by drying) the liquid component of a silica gel with air. They are generally synthesized via a sol-gel process in a liquid medium and then dried, usually by extraction with a supercritical fluid, the one most commonly used being supercritical CO₂. This type of drying makes it possible to avoid shrinkage of the pores and of the material.

Hydrophobic silica aerogel particles which may be used in the present invention can have a specific surface area per unit of mass (S_{M}) ranging from 500 to 1500 m²/g, preferably from 600 to 1200 m²/g and better still from 600 to 800 m²/g, and a size expressed as the volume-average diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles which may be used in the present invention have a size, expressed as volume-average diameter (D[0.5]), ranging from 1 to 30 μm, preferably from 5 to 25 μm, better still from 5 to 20 μm and even better still from 5 to 15 μm.

The sizes of the silica aerogel particles may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. According to one advantageous embodiment, the hydrophobic silica aerogel particles which may be used in the present invention have a specific surface area per unit of mass (S_{M}) ranging from 600 to 800 m²/g and a size expressed as the volume-average diameter (D[0.5]) ranging from 5 to 20 μm and even better still from 5 to 15 μm.

The silica aerogel particles which may be used in the present invention may advantageously have a tapped density ρ ranging from 0.02 g/cm³ to 0.10 g/cm³, preferably from 0.03 g/cm³ to 0.08 g/cm³ and preferably from 0.05 g/cm³ to 0.08 g/cm³.

According to one preferred embodiment, the hydrophobic silica aerogel particles which may be used in the present invention have a specific surface area per unit of volume S_{V} ranging from 5 to 60 m²/cm³, preferably from 10 to 50 m²/cm³ and better still from 15 to 40 m²/cm³. The specific surface area per unit of volume is given by the relationship: S_{V}=S_{M}×ρ, where ρ is the tapped density, expressed in g/cm³, and S_{M} is the specific surface area per unit of mass, expressed in m²/g, as defined above.

Preferably, the hydrophobic silica aerogel particles which may be used according to the invention have an oil-absorbing capacity, measured at the wet point, ranging from 5 to 18 ml/g, preferably from 6 to 15 ml/g and better still from 8 to 12 ml/g. The absorbing capacity measured at the wet point, noted Wp, corresponds to the amount of oil that needs to be added to 100 g of particles in order to obtain a homogeneous paste. It is measured according to the “wet point” method or the method for determining the oil uptake of a powder described in standard NF T 30-022. It corresponds to the amount of oil adsorbed onto the available surface of the powder and/or absorbed by the powder by measurement of the wet point, described below: An amount m=2 g of powder is placed on a glass plate, and the oil (isononyl isononanoate) is then added dropwise. After addition of 4 to 5 drops of oil to the powder, mixing is carried out using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

The aerogels which may be used according to the present invention are hydrophobic silica aerogels, preferably of silyl silica (INCI name: silica silylate). The term “hydrophobic silica” is understood to mean any silica of which the surface is treated with silylating agents, for example with halogenated silanes, such as alkylchlorosilanes, siloxanes, in particular dimethylsiloxanes, such as hexamethyldisiloxane, or silazanes, so as to functionalize the OH groups with silyl Si—Rn groups, for example trimethylsilyl groups.

The hydrophobic silica may be present in a concentration from about 0.25%, 0.5%, or 1% to about 1.5%, 2%, 3% or 5% by weight.

Polyhydric Alcohol

In accordance with certain embodiments of the present invention, compositions may include at least one polyhydric alcohol compound. The polyhydric alcohol compound may be selected from the group consisting of glycerin, glycols, polyglycerin, esters of polyhydric alcohols, and mixtures thereof.

Suitable glycols include those that are liquid at room temperature. The glycol may contain from 2 to 12 carbon atoms, such as from 2 to 10 carbon atoms such as, for example, glycerin, propylene glycol, butylene glycol, propane diol, hexylene glycol, polyglycerin, dipropylene glycol and diethylene glycol. In certain other embodiments the polyhydric alcohol compound includes glycerin, propylene glycol, butylene glycol, propane diol, hexylene glycol, polyglycerin, and combinations thereof. In certain other embodiments the polyhydric alcohol is glycerin.

Suitable esters of polyhydric alcohols include liquid esters of saturated or unsaturated, linear or branched C1-C26 polyhydric alcohols. The total number of carbon atoms of the esters may be greater than or equal to 8, such as greater than or equal to 10, such as greater than or equal to 12 and such as less than 50, including all ranges and subranges therebetween such as 8 to 50, 10 to 40, 12 to 30, 8 to 25, 10 to 50, etc. Specific examples of suitable esters of polyhydric alcohol include, but are not limited to, esters of dihydroxy, trihydroxy, tetrahydroxy or pentahydroxy alcohols. The ester of polyhydric alcohol may be a glyceryl ester such as, for example, glyceryl triglycolate, glyceryl tricitrate, glyceryl trilactate, glyceryl trilactate, glyceryl tributanoate, glyceryl triheptanoate, glyceryl trioctanoate, etc.

According to certain embodiments, the at least one polyhydric alcohol compound is present in the compositions of the present invention in an amount greater than about 3% by weight, such as greater than about 5% by weight. According to certain embodiments of the invention, the concentration of polydric alcohol compound is from about 3%, 5%, 8%, 10% or 12% to about 12%, 15%, 20%, 30%, 40% or 50%, with all weights being based on the weight of the composition.

C2-C4 Monoalcohol

According to certain embodiments of the invention, gel compositions of the present invention may include C2-C4 monoalcohol such as ethanol or isopropanol. The concentration of C2-C4 monoalcohol may range from about 0.5%, 1% or 2% to about 3%, 4%, 5% or 10%.

Other Fillers and Pigments

According to the present invention, gel compositions of the present invention may comprise other fillers and pigments (other than hydrophobic silica) that may be suspended in the gel composition.

The pigments which may be used according to the present invention may be chosen from white, colored, inorganic, organic, polymeric, and nonpolymeric pigments. Representative examples of mineral pigments include titanium dioxide, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

According to certain embodiments, other fillers and pigments are selected from those comprising iron oxide, titanium dioxide, and mica.

The other fillers and pigments useful in compositions of the present invention may be surface treated or not. For embodiments, in which the other filler or pigment is surface treated with a hydrophobic treatment, the concentrations of these hydrophobically treated fillers and pigments may be less than about 0.5%, such as less than about 0.1% by weight.

For other fillers and pigments that are not treated with a hydrophobic surface treatment, their collective concentration by weight in the gel compositions of the present invention may range from about 1%, 2%, 3%, or 5% to about 5%, 10%, or 20% by weight.

Aqueous Phase

The gel compositions of the present invention also contain water. Water is preferably present in an amount of from about 30%, 35%, 40%, or 50% to about 50%, 60%, 70%, 80% or 90% by weight, with all weights being based on the total weight of the composition.

Fatty Compounds

According to certain embodiments of the present invention, compositions may include at least one fatty compound (fatty substance). However, in certain other embodiments, it is desirable for the composition to be substantially free of such fatty compounds.

Fatty substances include oil(s) and/or wax(es). “Oil” means any non-aqueous medium which is liquid at ambient temperature (25° C.) and atmospheric pressure (760 mm Hg). A “wax” for the purposes of the present disclosure is a lipophilic fatty compound that is solid at ambient temperature (25° C.) and changes from the solid to the liquid state reversibly, having a melting temperature of more than 30° C. and, for example, more than 45° C., which can be as high as 150° C., a hardness of more than 0.5 MPa at ambient temperature, and an anisotropic crystalline organization in the solid state. By taking the wax to its melting temperature, it is possible to use wax(es) by themselves as carriers and/or it is possible to make wax(es) miscible with the oils to form a microscopically homogeneous mixture.

Suitable oils include volatile and/or non-volatile oils. Such oils can be any acceptable oil including but not limited to silicone oils and/or hydrocarbon oils.

According to certain embodiments, the compositions of the present invention preferably comprise one or more volatile silicone oils. Examples of such volatile silicone oils include linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils such as Octyltrimethicone, Hexyltrimethicone, Decamethylcyclopentasiloxane, Octamethylcyclotetrasiloxane, (cyclotetradimethylsiloxane or D4 cyclopentasiloxane or D5, and the like.

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexadecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are for example: Isododecane, Isohexadecane, Isopar L (isoparaffin C₁₁-C₁₃), Isopar H (isoparaffin C₁₁-C₁₂) and the like.

The volatility of the solvents/oils can be determined using the evaporation speed as set forth in U.S. Pat. No. 6,338,839, the contents of which are incorporated by reference herein.

According to certain embodiments of the present invention, the composition comprises at least one non-volatile oil. Examples of non-volatile oils that may be used in the present invention include, but are not limited to, polar oils such as: hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides

According to certain embodiments of the present invention, the compositions of the present invention further comprise at least one wax. Suitable examples of waxes that can be used in accordance with the present disclosure include those generally used in the cosmetics field: they include those of natural origin, such as beeswax, carnauba wax, candelilla wax, ouricoury wax, Japan wax, cork fibre wax or sugar cane wax, rice bran wax, rice wax, montan wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are concrete at 30° C., for example at 45° C.

According to certain embodiments of the present invention, the compositions of the present invention further include at least one silicone wax. Examples of suitable silicone waxes include, but are not limited to, silicone waxes such as alkyl- or alkoxydimethicones having an alkyl or alkoxy chain ranging from 10 to 4S carbon atoms, poly(di)methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

According to certain embodiments of the present invention, the compositions of the present invention further include at least one long-chain alcohol wax. Preferably, the at least one long-chain alcohol wax has an average carbon chain length of between about 20 and about 60 carbon atoms, most preferably between about 30 and about 50 carbon atoms. Suitable examples of long-chain alcohol waxes include but are not limited to alcohol waxes commercially available from Baker Hughes under the Performacol trade name such as, for example, Performacol 350, 425 and 550. Most preferably, the long-chain alcohol wax has a melting temperature range from about 93° C. to about 105° C.

According to preferred embodiments, the compositions of the present invention contain less than 2% wax and less than 2% oil.

According to preferred embodiments, the compositions of the present invention contain less than 1% wax and/or less than 1% oil.

According to preferred embodiments, the compositions of the present invention contain less than 0.5% wax and/or less than 0.5% oil.

According to preferred embodiments, the compositions of the present invention contain no wax and/or oil.

According to certain other embodiments, compositions of the present include up to about 2% volatile oil and less than about 1% of waxes and non-volatile oils.

Additional Additives

The composition of the invention can also comprise any additive usually used in the field under consideration. For example, dispersants such as poly(l2-hydroxystearic acid), antioxidants, essential oils, sunscreens, preserving agents, fragrances, fillers such as cellulose fibers, neutralizing agents, cosmetic and dermatological active agents such as, for example, emollients, moisturizers, vitamins, essential fatty acids, surfactants, silicone elastomers, thickening agents, gelling agents, particles, pasty compounds, viscosity increasing agents can be added. A non-exhaustive listing of such ingredients can be found in U.S. patent application publication no. 2004/0170586, the entire contents of which is hereby incorporated by reference. A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 70% (such as from 0.01% to 70%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the keratinous materials of human beings such as, for example, lips, skin or eyelashes.

According to preferred embodiments, the compositions of the present invention contain less than 1.5% surfactant. By “surfactant,” it is meant a compound having hydrophilic and hydrophobic portions and that when present in water in a concentration of 0.5%, is able to reduce surface tension of water at ambient temperature and pressure to less than about 60 mN/m, such as less than about 50 mN/m.

According to preferred embodiments, the compositions of the present invention contain less than 1.0% such as less than 0.5% surfactant.

According to certain other embodiments, gel compositions of the present invention include 20%-90% water, 3-40% polyhydric alcohol, 0.75% to 2% copolymer of sulfonic acid acrylic monomer, 1.75% to 4% of film-forming polymers selected from film-forming acrylic polymer, film-forming urethane polymer, and combinations thereof, and 0.5% to 2% hydrophobic silica.

According to preferred embodiments of the present invention, methods of treating, caring for and/or making up a keratinous material by applying compositions of the present invention to the keratinous material in an amount sufficient to treat, care for and/or make up the keratinous material are provided. Preferably, “making up” the keratinous material includes applying at least one coloring agent to the keratinous material in an amount sufficient to provide color to the keratinous material.

According to yet other preferred embodiments, methods of enhancing the appearance of a keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratinous material in an amount sufficient to treat, care for and/or make up the keratinous material, to cover or hide defects associated with keratinous material, or to enhance the appearance of keratinous material. The compositions may be applied to the desired area as needed, preferably once daily, and then preferably allowed to dry before subjecting to contact such as with clothing or other objects. Preferably, the composition is allowed to dry for about 4 minutes or less, more preferably for about 2 minutes or less.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1

The following are examples of compositions consistent with embodiments of the invention described herein:

| Ingredient | E1 | E2 | E3 |
|---|---|---|---|
| Water | 75-90 | 20-40 | 75-90 |
| Copolymer formed from sulfonic | 0.75-2.5 | 0.75-2.5 | 0.75-2.5 |
| acid acrylic monomer | | | |
| Film-Forming Acrylic Polymer | 1.5-2.0 | 1.5-2.0 | 1.5-2.0 |
| Film-Forming Urethane Polymer | 1.25-2.0 | 1.25-2.0 | 1.25-2.0 |
| Hydrophobic silica | 1.0-1.5 | 1.0-1.5 | 1.0-1.5 |
| Polyhydric alcohol compound | 3-10 | 30-40 | 3-10 |
| Additional pigments or fillers | 0 | 10-15 | 2-5 |
| C2-C4 Monoalcohol | 0 | 2-5 | 2-5 |
| Volatile oil | <0.5 | <1 | <0.5 |
| Surfactants and Emulsfiers | <1 | <1.5 | <1 |

### Example 2—Testing

Four compositions (Ex. 4, Ex. 5, Ex. 6 and Ex. 7, each prepared in duplicate) were prepared that were identical to each other, but with certain exceptions: Composition, Ex. 4 had only 0.72% copolymer formed from sulfonic acid acrylic monomer; Ex. 5 had only 0.2% copolymer formed from sulfonic acid acrylic monomer; Ex. 6 had no hydrophobic silica. Ex. 7 had 0.92% copolymer formed from sulfonic acid acrylic monomer and was consistent with E3 described above. All the samples included (brown) iron oxide as a portion of the additional pigment and filler. The varying percentages of copolymer were balanced by adjusting (q.s.) the concentrations of water. The samples were prepared by blending the ingredients under mixing and were then evaluated for stability.

Ex. 4 and Ex. 5 were allowed to equilibrate for a short period of time and were found to be phase unstable. Ex. 6 and Ex. 7 appeared stable, so they were subjected to a more rigorous “stress test” of accelerated stability test using a LumiSizer 651 available from LUM GmbH for multi-wavelength accelerated stability testing during centrifugation using a 10 mm path length and 865 nm source.

Specifically, the samples were subject to a rotational speed of 2000 rpm at 30 minutes at 45° C. and continually examined for the extinction of transmitted light across the full length of the sample. The samples were additionally subject to an even higher stress—3000 rpm at 30 minutes at 45° C.

Ex. 6 and 7 were examined after the centrifugation. Ex. 6 (no hydrophobic silica) separated into two distinct phases, a brown phase (presumably including colored pigments) on bottom and a white phase on top. Ex. 7 (including the hydrophobic silica) was uniform.

The LumiSizer 651 also calculates a sedimentation velocity, where higher values indicate faster sedimentation. The sedimentation velocities for each of the two replicates measured by the LumiSizer for Ex. 6 and Ex. 7 are shown in the table below:

| | Sedimentation | Sedimentation |
|---|---|---|
| | Velocity | Velocity |
| Reference | (Replicate 1) | (Replicate 2) |
| Ex. 6 | 0.201 | 0.304 |
| Ex. 7 | <0.001 | 0.013 |

Based on the centrifugation results and the sedimentation velocities, it can be seen that the presence of the hydrophobic silica is surprisingly important in maintaining stability of the gel compositions.

What is claimed is:

1. A gel composition, comprising:
water;
a water-soluble or water-dispersible gelling agent comprising at least one copolymer formed from a sulfonic acid acrylic monomer;
a film-forming polymer selected from a film-forming acrylic polymer, a film-forming urethane polymer, and combinations thereof; and
hydrophobic silica, wherein the gel composition is substantially free of fatty compounds.

2. The gel composition of claim 1, wherein the concentration by weight of the at least one copolymer formed from sulfonic acid acrylic monomer in the gel composition is from 0.75% to about 5%.

3. The gel composition of claim 1, wherein the water-soluble or water-dispersible gelling agent comprises a copolymer of 2-Acrylamido-2-methylpropane sulfonic acid and sodium acrylate, and a copolymer of 2-Acrylamido-2-methylpropane sulfonic acid and hydroxyethylacrylate.

4. The gel composition of claim 1, wherein the hydrophobic silica is present in the gel composition in a concentration by weight from about 0.5% to about 3%.

5. The gel composition of claim 1, wherein the hydrophobic silica is present in the gel composition in a concentration by weight from about 1% to about 3%.

6. The gel composition of claim 1, wherein the water is present in the gel composition in a concentration by weight in the gel composition from about 30% to about 90% by weight.

7. The gel composition of claim 1, wherein the water is present in the gel composition in a concentration by weight in the gel composition from about 30% to about 50% by weight.

8. The gel composition of claim 1, further comprising at least one polyhydric alcohol compound.

9. The gel composition of claim 1, wherein the at least one polyhydric alcohol compound is present in a concentration by weight in the gel composition from about 3% by weight to about 40% by weight.

10. The gel composition of claim 1, wherein the at least one polyhydric alcohol compound is present in a concentration by weight in the gel composition from about 12% by weight to about 40% by weight.

11. The gel composition of claim 1, wherein the gel composition comprises from about 0.5% to about 5% of at least one C2-C4 monoalcohol.

12. The gel composition of claim 1, wherein the gel composition comprises from about 5% by weight to about 20% by weight of additional pigments or fillers.

13. A method of treating, caring for and/or making up keratinous materials by applying the composition of claim 1 to the keratinous material of a user.
